(19) United States
(12) Patent Application Publication
(10) Pub. No.: US 2025/0018104 A1
(43) Pub. Date: Jan. 16, 2025

(54) A CATHETER SET FOR FORMING A FISTULA

(71) Applicant: CLEARSTREAM TECHNOLOGIES LIMITED, Enniscorthy, County Wexford (IE)

(72) Inventors: Sangeetha Rajendraprasad, Enniscorthy, Wexford (IE); Nicola Broaders, Enniscorthy, Wexford (IE); John O'Shea, Enniscorthy, Wexford (IE)

(21) Appl. No.: 18/711,810

(22) PCT Filed: Nov. 22, 2021
(86) PCT No.: PCT/EP2021/082459

§ 371 Date: May 20, 2024

(51) Int. Cl. A61M 1/36; A61B 17/34; A61M 27/00

(52) U.S. Cl. CPC A61M 1/3655; A61B 17/3415; A61M 27/002

(57) ABSTRACT

A catheter set for forming a fistula between two adjacent blood vessels. The catheter set comprises two crossing catheters. Each crossing catheter comprising a catheter shaft defining a longitudinal axis and having a distal end section, a stabilizing element disposed in the distal end section of the catheter shaft, and a penetration tool for penetrating vessel walls of two adjacent blood vessels to form the fistula. The penetration tool is disposed within the catheter shaft and is movable between a retracted position and an advanced position. The stabilizing element comprises an expandable cage having a contracted configuration and a laterally expanded configuration. The expandable cage is suitable for snaring a guidewire when moving from the laterally expanded configuration to the contracted configuration.

### TECHNICAL FIELD

The present disclosure relates to a catheter set for forming a fistula between two adjacent blood vessels, a method of forming a fistula between two adjacent blood vessels and a method of crossing a total chronic occlusion.

### BACKGROUND

Peripheral artery disease is a condition caused by abnormal narrowing of a peripheral arteries due to the deposit of atherosclerotic plaque in the walls of the arteries, thereby reducing or blocking blood flow to the limbs. In serious cases, peripheral artery disease can lead to complications such as infection, tissue death leading to amputation or stroke.

Peripheral artery disease can be treated in a number of ways such as by angioplasty or atherectomy. Another treatment option is to place a vascular bypass graft from a location proximal to the blockage to a location distal to the blockage in order to provide an unobstructed path for the blood to circumvent the diseased area. Such vascular bypass grafts are commonly placed in open vascular surgery which are highly invasive procedures and result in long recovery times for the patient. Another procedure used to treat peripheral artery disease is a deep vein arterialization procedure, where a fistula is created between a peripheral artery and vein with the intent of “arterializing” the veins. Arterial blood flows through the veins and bypasses the blockage in the artery to thereby provides adequate blood flow to the target tissue.

US 2014/0142677 A1 discloses a system and method for placing a vascular bypass graft in an endovascular procedure, making the procedure less invasive. The system comprises a penetration catheter carrying a penetration tool for penetrating from a peripheral artery to a peripheral vein.

The system further comprises a guidewire capture and stabilization catheter which comprises a braid and is designed to capture a guidewire from the penetration catheter in the peripheral vein. The penetration catheter also comprises a stabilizing element in the form of a balloon, expandable braid or malecot.

However, the system and method of US 2014/0142677 A1 require a large number of steps and multiple specialised catheters to perform the endovascular bypass procedure, which prolongs the duration of the procedure. Furthermore, the system is limited in its flexibility to cross between the artery and vein at different points above and below a chronic total occlusion.

There is hence a need in the art for a new type of catheter system, which can perform a deep vein arterialization or endovascular bypass procedure in a more efficient manner, requiring fewer steps and fewer catheters. There is also a need for a new type of catheter system which can be used to form fistulas in a more flexible manner to treat peripheral artery disease.

### SUMMARY

In a first aspect of the present disclosure, there is provided a catheter set for forming a fistula between two adjacent blood vessels. The catheter set comprises two crossing catheters. Each crossing catheter comprises a catheter shaft defining a longitudinal axis and having a distal end section, a stabilizing element disposed in the distal end section of the catheter shaft, and a penetration tool for penetrating vessel walls of two adjacent blood vessels to form the fistula. The penetration tool is disposed within the catheter shaft and is movable between a retracted position and an advanced position. The stabilizing element comprises an expandable cage having a contracted configuration and a laterally expanded configuration. The expandable cage is suitable for snaring a guidewire when moving from the laterally expanded configuration to the contracted configuration.

In some embodiments, this may result in a catheter system which can perform a deep vein arterialization or endovascular bypass procedure in a more efficient manner, requiring fewer steps.

Throughout this disclosure the term ‘fistula’ is used to denote a connection or passageway.

Throughout this disclosure the term ‘crossing catheter’ is used to denote a catheter which is suitable for forming a fistula in a vessel.

Throughout this disclosure, the term ‘to snare’ means ‘to catch’ or ‘to trap’.

At least one, optionally both, of the stabilizing elements may further comprise a mesh or a screen which covers the expandable cage.

In some embodiments, this may result in the stabilizing element being able to better snare or catch a guidewire.

In some embodiments, this may also result in a stabilizing element which can better expand the size of the fistula.

The mesh or screen may have an antithrombic coating.

In some embodiments, this may reduce the formation of blood clots.

At least one, optionally both, of the expandable cages comprise a plurality of flexible spines.

The flexible spines may be longitudinally oriented.

The expandable cage may comprise 3 to 10, optionally, 3 to 5 flexible spines.

In some embodiments, this may allow the expandable cage to easily expand and collapse.

In some embodiments, this may further result in the stabilizing element being able to better snare or catch a guidewire.

At least one, optionally both, of the expandable cages may comprise a radiopaque marker.

In some embodiments, this may allow the crossing catheters to be more accurately aligned.

The radiopaque marker may be disposed at a centre portion of the expandable cage.

In some embodiments, this may allow the crossing catheters to be more accurately aligned.

At least one, optionally both, of the two crossing catheters may comprise a guidewire lumen for accommodating a guidewire.

In some embodiments, this may allow the crossing catheter to be more easily guided to a target location over a guidewire.

The guidewire lumen may be disposed within the penetration tool, such that the guidewire lumen allows placement of a guidewire through the fistula formed by the penetration tool.

In some embodiments, this may allow quick and simple placement of a guidewire directly through the fistula.

The catheter set may further comprise a guidewire disposed within the guidewire lumen, wherein the guidewire comprises a radiopaque marker disposed at its distal end.

In some embodiments, this may help to better align the distal tip of the guidewire with the stabilizing element of the crossing catheter in the other vessel which may result in better snaring or catching of the guidewire.

At least one, optionally both, of the catheter shafts may further comprise a second guidewire lumen for accommodating a second guidewire.

In some embodiments, this may allow the crossing catheter to be more easily guided to a target location over a guidewire.

The catheter set may further comprise a second guidewire disposed within the second guidewire lumen, wherein the second guidewire has a radiopaque marker disposed at its distal end.

Each catheter shaft may comprise a penetration lumen for accommodating the penetration tool.

In some embodiments, this may allow the penetration tool to be accommodated within the catheter shaft.

The distal end of each penetration lumen may end in a penetration port through which the penetration tool can exit the crossing catheter.

At least one, optionally both, of the penetration ports may be disposed at a distal end of the respective crossing catheter.

In some embodiments, this may allow the crossing catheter to more easily translated through the fistula.

At least one, optionally both, of the crossing catheters may comprise a tapered distal tip. The penetration port may be disposed at the distal end of the tapered distal tip.

In some embodiments, this may allow the crossing catheter to enlarge the size of the fistula, such that the crossing catheter can be translated through the fistula.

At least one, optionally both, of the penetration ports may be disposed adjacent the stabilizing element.

In some embodiments, this may result in better stabilization of the crossing catheter during the fistula forming process.

At least one, optionally both, of the penetration ports may be disposed distally adjacent the stabilizing element.

At least one, optionally both, of the penetration ports may be disposed proximally adjacent the stabilizing element.

At least one, optionally both, of the penetration ports may be disposed within the stabilizing element.

In some embodiments, this may result in better stabilization of the crossing catheter during the fistula forming process.

The stabilizing element may be a first stabilizing element and at least one, optionally both, of the crossing catheters may further comprise a second stabilizing element.

In some embodiments, this may result in better stabilization of the crossing catheter during the fistula forming process.

In some embodiments, this may also allow easier snaring of a guidewire using the crossing catheter.

The penetration port may be disposed between the first stabilizing element and the second stabilizing element.

The second stabilizing element may comprise an expandable cage having a contracted configuration and a laterally expanded configuration.

The second stabilizing element may be identical to the first stabilizing element.

The second stabilizing element may further comprise a mesh or a screen which covers the expandable cage.

The mesh or screen may have an antithrombic coating.

The expandable cage of the second stabilizing element may comprise a plurality of flexible spines.

The spines may be longitudinally oriented.

At least one, optionally both, of the penetration ports may comprise a lateral opening in the catheter shaft.

At least one, optionally both, of the penetration ports may comprise a longitudinal opening in the catheter shaft.

A distal end of at least one, optionally both, of the penetration tools deflect laterally as the penetration tool is moved distally from the retracted position to the advanced position.

In some embodiments, this may allow the penetration tool to more easily penetrate and form a fistula in a vessel wall.

At least one, optionally both, of the penetration tools may comprise a needle.

In a second aspect of the present disclosure, there is provided a crossing catheter for forming a fistula between two adjacent blood vessels. The crossing catheter comprises a catheter shaft defining a longitudinal axis and having a distal end section, a stabilizing element disposed in the distal end section of the catheter shaft, and a penetration tool for penetrating vessel walls of two adjacent blood vessels to form the fistula. The penetration tool is disposed within the catheter shaft and is movable between a retracted position and an advanced position. The stabilizing element comprises an expandable cage having a contracted configuration and a laterally expanded configuration. The expandable cage is suitable for snaring a guidewire when moving from the laterally expanded configuration to the contracted configuration.

In some embodiments, this may result in a crossing catheter which can perform a deep vein arterialization or endovascular bypass procedure in a simpler manner and requiring fewer steps.

The stabilizing element may further comprise a mesh or a screen which covers the expandable cage.

In some embodiments, this may result in the stabilizing element being able to better snare or catch a guidewire.

In some embodiments, this may also result in a stabilizing element which can better expand the size of the fistula.

The mesh or screen may have an antithrombic coating.

In some embodiments, this may reduce the formation of blood clots.

The expandable cage may comprise a plurality of flexible spines.

The plurality of flexible spines may be longitudinally oriented.

The expandable cage may comprise 3 to 10, or for example, 3 to 5 flexible spines.

In some embodiments, this may allow the expandable cage to easily expand and collapse.

In some embodiments, this may further result in the stabilizing element being able to better snare or catch a guidewire.

The expandable cage may comprise a radiopaque marker.

In some embodiments, this may allow the crossing catheters to be more accurately aligned.

The radiopaque marker may be disposed at a centre portion of the expandable cage.

In some embodiments, this may allow the crossing catheters to be more accurately aligned.

The crossing catheter may further comprise a guidewire lumen for accommodating a guidewire.

In some embodiments, this may allow the crossing catheter to be more easily guided to a target location over a guidewire.

The guidewire lumen may be disposed within the penetration tool, such that the guidewire lumen allows placement of a guidewire through the fistula formed by the penetration tool.

In some embodiments, this may allow quick and simple placement of a guidewire directly through the fistula.

The crossing catheter may further comprise a guidewire disposed within the guidewire lumen. The guidewire may comprise a radiopaque marker disposed at its distal end.

In some embodiments, this may help to better align the distal tip of the guidewire with the stabilizing element of the crossing catheter in the other vessel which may result in better snaring or catching of the guidewire

The catheter shaft may further comprise a second guidewire lumen for accommodating a second guidewire.

In some embodiments, this may allow the crossing catheter to be more easily guided to a target location over a guidewire.

The crossing catheter may further comprise a second guidewire disposed within the second guidewire lumen. The second guidewire may have a radiopaque marker disposed at its distal end.

In some embodiments, this may allow easier and more accurate positioning of the guidewire.

The catheter shaft may comprise a penetration lumen for accommodating the penetration tool.

In some embodiments, this may allow the penetration tool to be accommodated within the catheter shaft.

The distal end of the penetration lumen may end in a penetration port through which the penetration tool can exit the crossing catheter.

The penetration port may be disposed at the distal end of the crossing catheter.

In some embodiments, this may allow the crossing catheter to more easily translated through the fistula.

The crossing catheter may comprise a tapered distal tip. The penetration port may be disposed at the distal end of the tapered distal tip.

In some embodiments, this may allow the crossing catheter to enlarge the size of the fistula, such that the crossing catheter can be translated through the fistula.

The penetration port may be disposed adjacent the stabilizing element.

In some embodiments, this may result in better stabilization of the crossing catheter during the fistula forming process.

The penetration port may be disposed distally adjacent the stabilizing element.

The penetration port may be disposed proximally adjacent the stabilizing element.

The penetration port may be disposed within the stabilizing element.

In some embodiments, this may result in better stabilization of the crossing catheter during the fistula forming process.

The stabilizing element may be a first stabilizing element and the crossing catheter may further comprise a second stabilizing element.

In some embodiments, this may result in better stabilization of the crossing catheter during the fistula forming process.

The second stabilizing element may be identical to the first stabilizing element.

The second stabilizing element may further comprise a mesh or a screen which covers the expandable cage.

The mesh or screen may have an antithrombic coating.

The expandable cage of the second stabilizing element may comprise a plurality of flexible spines.

The plurality of flexible spines may be longitudinally oriented.

The penetration port may be disposed between the first stabilizing element and the second stabilizing element.

The second stabilizing element may comprise an expandable cage having a contracted configuration and a laterally expanded configuration.

The penetration port may comprise a lateral opening in the catheter shaft.

The penetration port may comprise a longitudinal opening in the catheter shaft.

A distal end of the penetration tool may deflect laterally as the penetration tool is moved distally from the retracted position to the advanced position.

In some embodiments, this may allow the penetration tool to more easily penetrate and form a fistula in a vessel wall.

The penetration tool may comprise a needle.

In a third aspect of the present disclosure, there is provided a method of forming a fistula between two adjacent blood vessels using a first and second crossing catheter, each crossing catheter comprising a catheter shaft defining a longitudinal axis and having a distal end section, a stabilizing element comprising an expandable cage disposed in the distal end section of the catheter shaft, and a penetration tool disposed within the catheter shaft, the method comprising introducing the first crossing catheter into a first blood vessel. Introducing the second crossing catheter into a second adjacent blood vessel. Advancing the first and second crossing catheters to a predetermined crossing point. Stabilizing the first crossing catheter by moving the stabilizing element of the first crossing catheter from a contracted configuration to a laterally expanded configuration. Moving the stabilizing element of the second crossing catheter from a contracted configuration to a laterally expanded configuration. Forming the fistula by moving the penetration tool of the first crossing catheter from a retracted position to an advanced position and penetrating vessel walls between the two adjacent blood vessels. Passing a guidewire through the fistula from the first blood vessel to the second blood vessel. Snaring the guidewire with the stabilizing element of the second crossing catheter by moving from the laterally expanded configuration to the contracted configuration.

In some embodiments this may result in a simpler and more flexible method of forming a fistula.

In a fourth aspect of the present disclosure, there is provided a method for bypassing a total chronic occlusion in a blood vessel using a first and second crossing catheter. Each crossing catheter comprises a catheter shaft defining a longitudinal axis and having a distal end section, a stabilizing element comprising an expandable cage disposed in the distal end section of the catheter shaft, and a penetration tool disposed within the catheter shaft. The method comprises: introducing the first crossing catheter into the blood vessel comprising the total chronic occlusion from a first side. Advancing the first crossing catheter to a first position adjacent the chronic total occlusion.

Stabilizing the first crossing catheter by moving the stabilizing element of the first crossing catheter from a contracted configuration to a laterally expanded configuration. Forming a first fistula between the blood vessel and an adjacent blood vessel by moving the penetration tool of the first crossing catheter from a retracted position to an advanced position and penetrating vessel walls between the two adjacent blood vessels. Moving the stabilizing element of the first crossing catheter from the laterally expanded configuration to the contracted configuration.

Translating the first crossing catheter through the first fistula into the adjacent blood vessel. Introducing the second crossing catheter into the blood vessel comprising the total chronic occlusion from a second side. Advancing the second crossing catheter to a second position adjacent the chronic total occlusion on the opposite side to the first position. Moving the stabilizing element of the first crossing catheter from a contracted configuration to a laterally expanded configuration. Stabilizing the second crossing catheter by moving the stabilizing element of the second crossing catheter from a contracted configuration to a laterally expanded configuration. Forming a second fistula between the blood vessel and the adjacent blood vessel by moving the penetration tool of the second crossing catheter from a retracted position to an advanced position and penetrating vessel walls between the two adjacent blood vessels. Passing a guidewire through the second fistula from the first blood vessel to the second blood vessel. Snaring the guidewire with the stabilizing element of the first crossing catheter by moving from the laterally expanded configuration to the contracted configuration. Pulling the first crossing catheter and the guidewire through the first fistula into the blood vessel.

In some embodiments, this may result in a simpler endovascular bypass procedure requiring fewer steps.

Translating the first crossing catheter through the first fistula may further comprise expanding the size of the first fistula by moving the stabilizing element of the first crossing catheter from a contracted configuration to a laterally expanded configuration.

Translating the second crossing catheter through the second fistula may further comprise expanding the size of the second fistula by moving the stabilizing element of the second crossing catheter from a contracted configuration to a laterally expanded configuration.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1A shows a side view of a distal section of a crossing catheter according to an embodiment of the present disclosure in a contracted configuration.

FIG. 1B shows a proximal section of the crossing catheter of FIG. 1A.

FIG. 2 shows a side view of the crossing catheter of FIG. 1 in an expanded configuration.

FIGS. 3A-3L illustrate a method of performing an endovascular bypass procedure using two of the crossing catheters of FIG. 1.

FIGS. 4A and B show a side view of an alternative embodiment of a crossing catheter according to the present disclosure in a contracted and an expanded configuration.

FIGS. 5A and B show a side view of an alternative embodiment of a crossing catheter according to the present disclosure in a contracted and expanded configuration.

FIGS. 6A and B show a side view of an alternative embodiment of a crossing catheter according to the present disclosure in a contracted and expanded configuration.

FIGS. 7A and B show a side view of an alternative embodiment of a crossing catheter according to the present disclosure in a contracted and expanded configuration.

FIGS. 8A-C illustrate a method of destroying a venous valve using the crossing catheter of FIG. 5.

FIGS. 9A-F illustrate a method of performing a deep vein arterialization procedure using two of the crossing catheters of FIG. 5.

### DETAILED DESCRIPTION

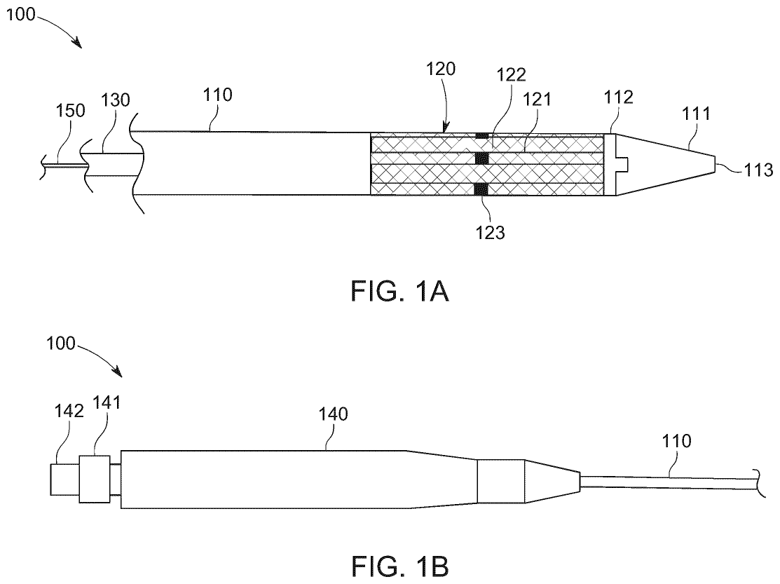

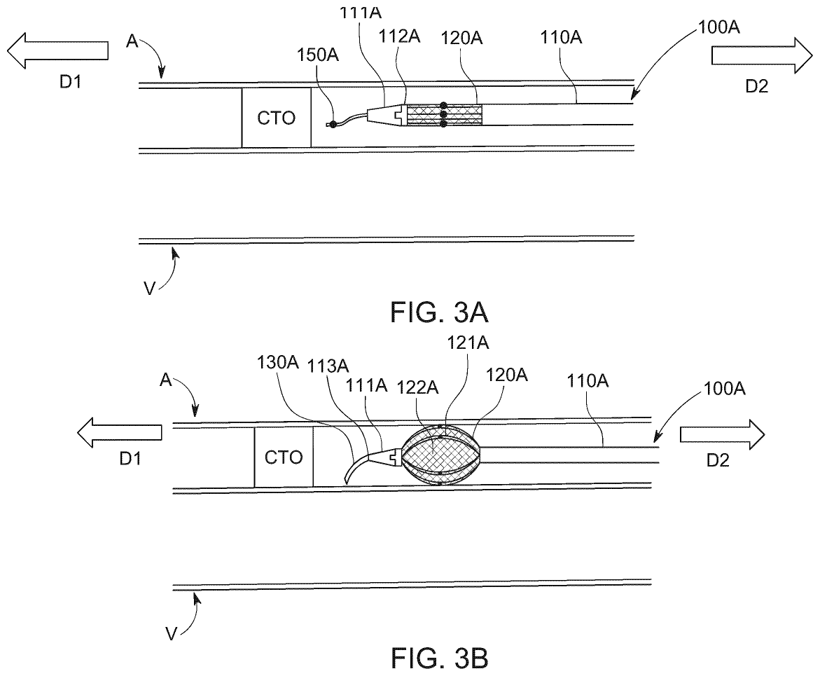

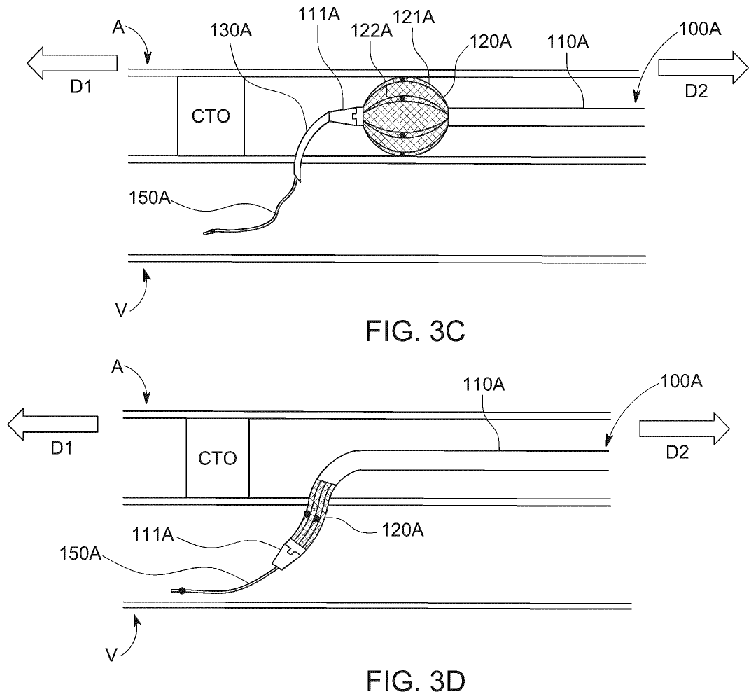

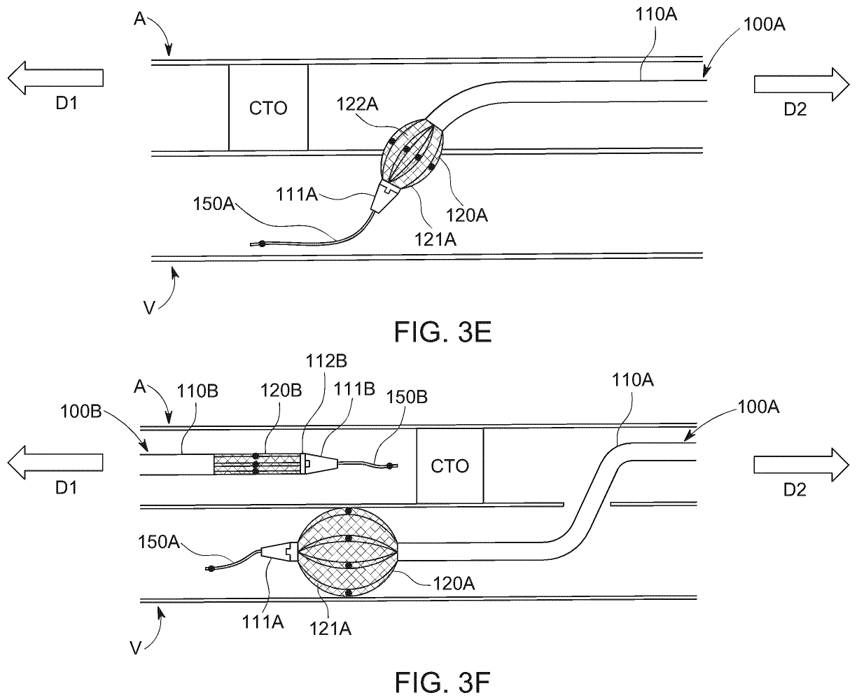

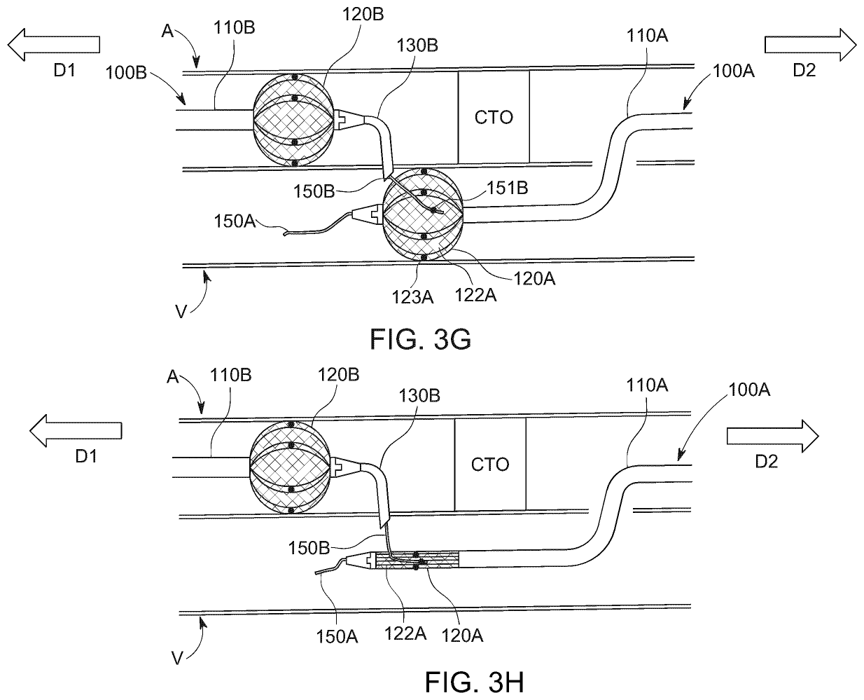

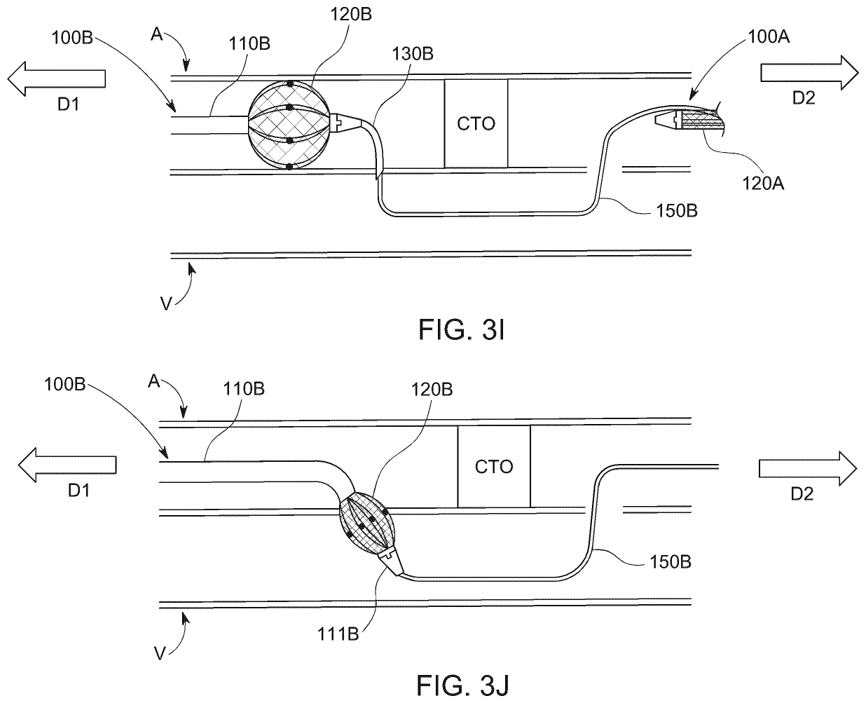

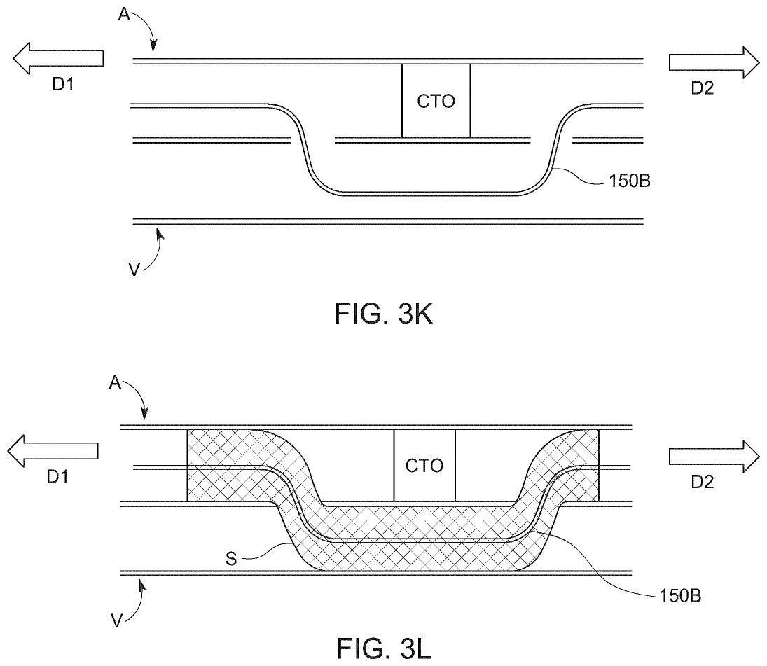

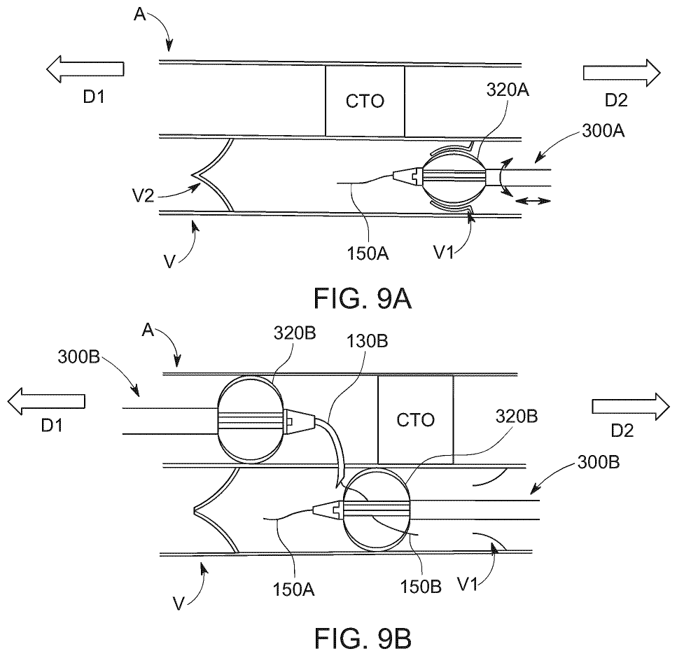

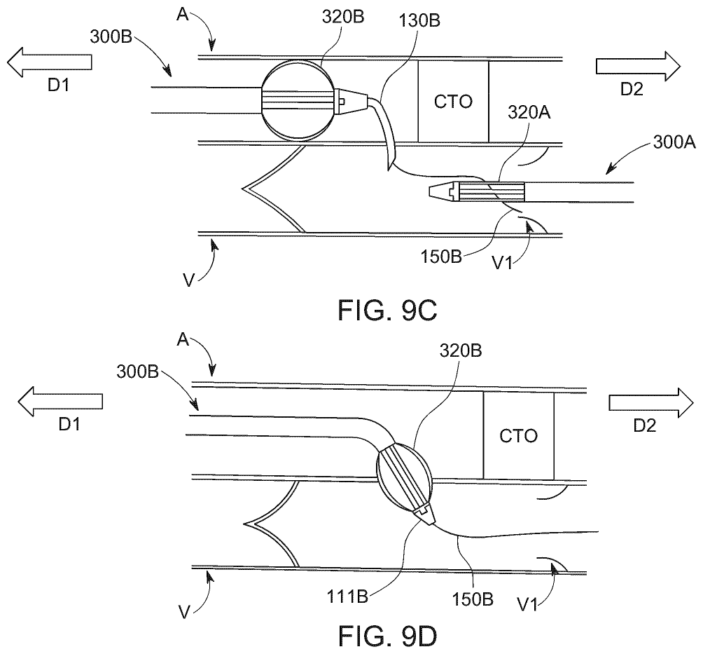

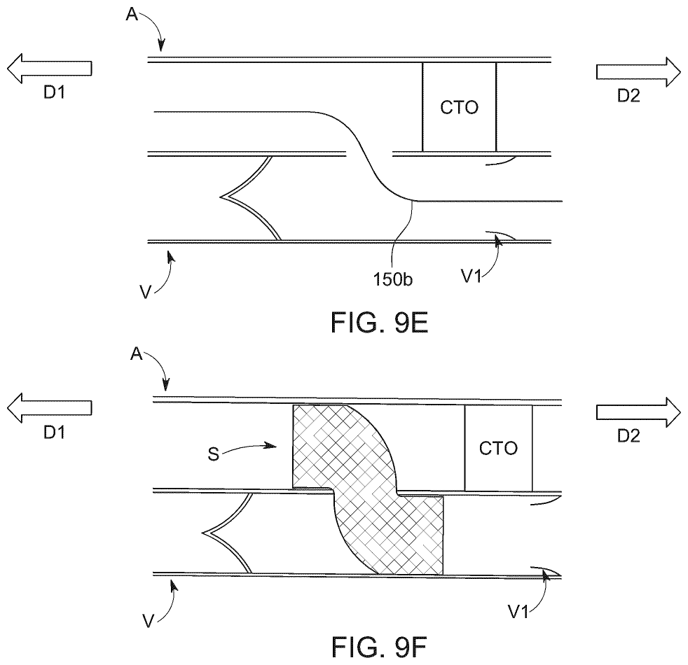

FIGS. 1A and B show an embodiment of a crossing catheter 100 for forming a fistula. Two crossing catheters 100 can be used to perform an endovascular bypass procedure or a deep vein arterialization procedure, as explained with respect to FIG. 3 and FIG. 9 below.

FIG. 1A shows a distal section of the crossing catheter 100. The crossing catheter 100 comprises a catheter shaft 110 with a stabilizing element 120 disposed in the distal end section of the catheter shaft 110. A tapered distal tip 111 may be disposed at the distal end of the crossing catheter. The tapered distal tip 111 tapers towards a distal end, i.e. the diameter of the tapered distal tip 111 decreases towards the distal end. This tapered shape allows the crossing catheter 100 to dilate a fistula such that the crossing catheter 100 can be translated through the fistula.

The crossing catheter 100 further comprises a penetration tool 130 for penetrating a vessel wall and forming the fistula. The penetration tool 130 may be slidably disposed within a penetration lumen and can exit the crossing catheter 100 through a penetration port 113 at the distal end of the tapered distal tip 111. FIG. 1A shows the penetration tool 130 in the retracted position where the penetration tool 130 is fully disposed within the penetration lumen and does not extend from the penetration port 113.

A radiopaque marker 112 may be positioned between the stabilizing element 120 and the tapered distal tip 111 to allow the position of the stabilizing element 120 and the tapered distal tip 111 to be accurately determined under fluoroscopy.

The stabilizing element 120 is in the form of an expandable cage which has a contracted configuration and a laterally expanded configuration. FIG. 1A shows the stabilizing element 120 in the contracted configuration in which the radial extent of the stabilizing element is smaller than in the laterally expanded configuration. The contracted configuration allows the crossing catheter 100 to be introduced and moved along a blood vessel to a target location, while the laterally expanded configuration allows the stabilizing element 120 to stabilize the crossing catheter 100 during a fistula formation process. The stabilizing element 120 may comprise multiple flexible spines 121. In the contracted configuration of FIG. 1A the flexible spines 121 lie flat in a longitudinal orientation. In the embodiment of FIG. 1A, the crossing catheter 100 has 4 flexible spines 121, however, the crossing catheter 100 may comprise fewer or more flexible spines 121, for example, in the range of 3 to 10 flexible spines, or for example 3 to 5 flexible spines 121. A mesh or screen 122 may cover the flexible spines 121. The mesh or screen 122 helps the stabilizing element 121 to better snare or catch a guidewire.

It also allows the stabilizing element 120 to more effectively expand the diameter of the fistula. The mesh or screen 122 may have an antithrombic coating to reduce the risk of blood clotting.

Each of the flexible spines 121 may comprise a radiopaque marker 123, which is disposed in the centre of the stabilizing element 120. The radiopaque markers 123 may allow the position of the stabilizing element to be more accurately determined and thereby help in correctly positioning the stabilizing element 120 when forming a fistula or snaring a guidewire.

The crossing catheter 100 may further comprise a guidewire 150 which is disposed in a guidewire lumen within the penetration tool 130 and may exit the guidewire lumen through the distal tip of the penetration tool 130. When the penetration tool 130 is in the retracted position, the guidewire 150 may be used as a placement guidewire for conventional over-the-wire placement to guide the crossing catheter 100 to an appropriate treatment site. When the penetration tool 130 is in the advanced position, the guidewire 150 can be directly placed through a fistula formed by the penetration tool 130.

In an alternative embodiment, the crossing catheter 100 may comprise a second guidewire lumen within the catheter shaft 110 accommodating a second guidewire, in addition to guidewire 150. The second guidewire may be used as a placement guidewire, whereas the guidewire 150 is arranged to be placed through a fistula formed by the penetration tool 130.

FIG. 1B shows a proximal section of the crossing catheter 100. A handle 140 may be disposed at the proximal end of the catheter shaft 110. The handle 140 comprises a number of mechanisms of a type which are conventionally employed in catheter construction and are not described in detail here.

The handle 140 may comprise a penetration mechanism 140 which allows a physician to move the penetration tool 130 longitudinally within the penetration lumen. This allows a physician to move the penetration tool from a retracted position where the penetration tool 130 is fully disposed within the penetration lumen to an advanced position where the penetration tool 130 extends out of the penetration port 113 such that it can penetrate a vessel wall. The handle 140 may also include a mechanism for expanding and contracting the stabilizing element 120. This may be done, for example, through a push-pull wire mechanism in the handle 140.

Furthermore, the handle 140 may comprise a guidewire advancement mechanism 142 for advancing and retracting the guidewire 150 within the guidewire lumen of the penetration tool 130. This allows the guidewire 150 to be advanced directly through a fistula formed by the penetration tool 130.

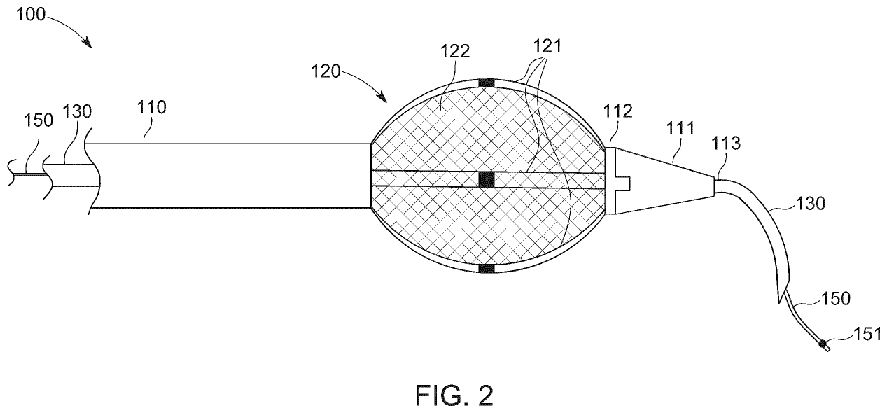

FIG. 2 shows a side view of the distal section of the crossing catheter 100 with the stabilizing element 120 in the laterally expanded configuration. In the laterally expanded configuration, the flexible spines 121 of the stabilizing element 120 remain in a longitudinal orientation but may be bent into a convex shape such that they have a greater lateral or radial extent than in the contracted configuration. This allows the stabilizing element 120 to contact the inside of a vessel wall and thereby stabilize the crossing catheter 100 such that it cannot move laterally in the vessel. To that extent, the flexible spines 121 bend when the tapered distal tip 111 is moved proximally with respect to the catheter shaft 110. This may be achieved, for example, through the use of a pull wire which is attached to the tapered distal tip 111 and may be operated using a mechanism in the handle 140 to move the stabilizing element 120 from the contracted configuration to the laterally expanded configuration. The amount of expansion of the stabilizing element 120 may be controlled by adjusting the distance that the pull wire is moved proximally.

The mesh or screen 122 may also cover the flexible spines 121 in the laterally expanded configuration. As will be explained below with reference to FIG. 3, the stabilizing element 120 can be inserted into the newly formed fistula and expanded to dilate the fistula. The mesh or screen 122 allows the fistula to be expanded more evenly and effectively.

FIG. 2 also shows the penetration tool 130 in the advanced position where it extends from the penetration port 113 at the distal end of the tapered distal tip 111. As noted above, the advancement of the penetration tool 130 can be controlled through a penetration mechanism 141 in the handle 140. The penetration tool 130 may be a curved, resilient needle with a sharp distal tip which deploys over a radially out-ward curved path as it is advanced to penetrate the vessel wall.

In other words, the penetration tool 130 may deflect laterally as the penetration tool 130 is moved distally from the retracted position to the advanced position. This may be achieved by forming a pre-bent portion in the penetration tool 130 or making the penetration tool 130 from a shape memory material such as Nitinol.

The guidewire 150 is shown in FIG. 2 as advancing out of the distal end of the penetration tool 130. With the penetration tool 130 in the advanced position, the guidewire 150 may be placed directly through a fistula formed by the penetration tool 130.

The guidewire 150 may comprise a radiopaque marker 151 at or near its distal end to allow it to be accurately located under fluoroscopy.

FIGS. 3A to 3L illustrate a method of using two crossing catheters 100A and 100B to perform an endovascular bypass procedure. The crossing catheters 100A and 100B both correspond to the crossing catheter 100 described with reference to FIGS. 1 and 2, and are suitable for use in both the arterial and venous system.

A chronic total occlusion CTO is the complete or nearly complete blockage of an artery caused by the build-up of atherosclerotic plaque. In the case of a blockage in a peripheral artery, this can reduce blood flow to the limbs resulting in peripheral artery disease. In an endovascular bypass procedure, a vascular graft is placed to bypass the chronic total occlusion CTO and provide an unobstructed path for the blood to circumvent the diseased area.

Firstly, a suitable access site is formed in an artery A, and a first guidewire 150A is introduced into the artery A through the access site and advanced to the CTO under fluoroscopy in a first direction D1.

For an endovascular bypass procedure, the access site may be distal of the CTO such that the first direction D1 is a proximal direction and the second direction D2 is a distal direction. If the CTO is in located in an artery of the leg, for example, then the access site would be below the CTO and the first guidewire 150A would approach the CTO from below.

As shown in FIG. 3A, a first crossing catheter 100A together with the penetration tool 130A is then introduced over the guidewire 150A into the artery A through the access site. The crossing catheter 100A is then advanced along the guidewire 150A in a first direction D1 to a first side of the CTO.

During advancement of the first crossing catheter 100A, the stabilizing element 120A is in the contracted configuration.

The radiopaque marker 112A helps to accurately identify the location of the distal tip 111A and the stabilizing element 120A under fluoroscopy.

A suitable crossing point may be determined, for example, by viewing the site of the CTO under fluoroscopy. Alternatively, the penetration tool 130A may be partially advanced and rotated such that the distal end faces the vein V. The guidewire 150A may then be advanced through the guidewire lumen of the penetration tool 130A until it extends from the distal end of the penetration tool 130A. The physician can then feel the wall of the artery A using the guidewire 150A to determine a suitable crossing point for forming the fistula where the vessel wall is less calcified.

As shown in FIG. 3B, once the first crossing catheter 100A is positioned adjacent the CTO, and a suitable crossing point for crossing over into the adjacent vein V has been identified, the stabilizing element 120A is expanded from the contracted configuration into the laterally expanded configuration such that it contacts the wall of the artery A.

The guidewire 150A is retracted and the penetration tool 130A is advanced such that it extends out of the penetration port 113A at the distal end of the tapered distal tip 111A. The sharp distal needle end of the penetration tool 130A deflects laterally and penetrates the vessel wall of the artery A and vein V to form a connection or fistula between the artery A and vein V, on a first side of the CTO. During the fistula formation, the stabilizing element 120B is in the laterally expanded configuration and thereby prevents lateral movement of the crossing catheter 100A and, specifically, the tapered distal tip 111A to allow the penetration tool 130A to penetrate the vessel walls.

As shown in FIG. 3C, once the fistula has been formed and the penetration tool 130A has reached the lumen of the vein V, the guidewire 150A is advanced through the guidewire lumen in the penetration tool 130A and through the fistula directly into the vein V.

The penetration tool 130A is then retracted and the stabilizing element 120A is collapsed into the contracted configuration. The guidewire 150A remains positioned through the fistula in the lumen of the vein V. The crossing catheter 100A is then advanced in the first direction D1 along the guidewire 150A through the fistula into the lumen of the vein V. The tapered shape of the tapered distal tip 111A helps to widen the fistula to allow the crossing catheter 100A to pass into the fistula.

FIG. 3D shows the stabilizing element 120A positioned within the fistula. At that point, the stabilizing element 120A is expanded to expand the size of the fistula, as shown in FIG. 3E. The mesh or screen 122A ensures a more even expansion of the fistula and prevents damage to the vessel wall from the flexible spines 121A. This avoids the need for a separate balloon catheter to be introduced to enlarge the fistula and significantly simplifies and shortens the procedure.

The procedure continues and the first crossing catheter 100A is further advanced in a first direction D1 along the guidewire 150A within the lumen of the vein V past the CTO.

Meanwhile, a second access site is formed in the artery A on the opposite side of the CTO. A second guidewire 150B is introduced into the artery A and advanced to the CTO under fluoroscopy in a second direction D2. A second crossing catheter 100B is introduced over the guidewire 150B into the artery A from the second access site and advanced in the second direction D2 to a position adjacent the CTO on a second side of the CTO. A suitable crossing point on the second side of the CTO is determined.

As shown in FIG. 3F, the stabilizing element 120A of the first crossing catheter is then expanded into the laterally expanded configuration to stabilize the first crossing catheter 100A in the vein V, opposite the pre-determined crossing point.

The stabilizing element 120B of the second crossing catheter 120B is then expanded to stabilize the crossing catheter 100B for the fistula formation process. The guidewire 150B is retracted and the penetration tool 130B is advanced such that it extends out of the penetration port 113B at the distal end of the tapered distal tip 111B of the second crossing catheter 100B. The sharp distal needle end of the penetration tool 130B deflects laterally and penetrates the vessel wall of the artery A and vein V to form a second connection or fistula between the artery A and vein V, on the second side of the CTO.

Once the second fistula has been formed, and the penetration tool 130B has reached the lumen of the vein V, the guidewire 150B is advanced through the guidewire lumen in the penetration tool 130B and through the fistula directly into the vein V.

As shown in FIG. 3G, the expanded stabilizing element 120A of the first crossing catheter 100A is positioned in the vein V next to the second fistula. When the guidewire 150B is advanced into the vein V, it moves through the mesh 122A and past the flexible spines 121A into the stabilizing element 120A. The mesh or screen 122A comprises apertures which allow the guidewire 150B to advance through the mesh or screen 122A and into the stabilizing element 120A. The radiopaque marker 151B on the guidewire 150B and the radiopaque markers 123A on the stabilizing element 120A allow an accurate visualisation of the relative position of the guidewire 150B and the stabilizing element 120A under fluoroscopy.

When the radiopaque marker 151B is either aligned with the radiopaque markers 123A or has moved past the radiopaque markers 123A, then the stabilizing element 120A is collapsed from the laterally expanded configuration to the contracted configuration to snare or capture the guidewire 150B, as shown in FIG. 3H. The mesh or screen 122A helps to securely snare or capture the guidewire 151A.

As shown in FIG. 3I, the first crossing catheter 100A, together with the snared guidewire 150B, is then pulled in a second direction D2 back through the first fistula and may be pulled all the way out of the first access site.

The penetration tool 130B of the second crossing catheter 100B is then retracted and the stabilizing element 120B is collapsed into the contracted configuration. The second crossing catheter 100B is then advanced along the guidewire 150B through the second fistula partly into the lumen of the vein V. The tapered shape of the distal tip 111B helps to widen the second fistula to allow the second crossing catheter 100B to pass into the second fistula.

When the stabilizing element 120B positioned within the fistula, as shown in FIG. 3J, the stabilizing element 120B is expanded to expand the size of the second fistula. The mesh or screen 122B ensures a more even expansion of the fistula and prevents damage to the vessel wall from the flexible spines 121B.

The stabilizing element 120B is then collapsed into the contracted configuration and the second crossing catheter 100B is pulled back in a first direction D1 out of the second access site, such that only the guidewire 150B is left, as illustrated in FIG. 3K. The guidewire 150B extends from the first access site through the first fistula into the vein V, past the CTO, through the second fistula back into the artery A and out of the second access site.

A standard balloon catheter with a stent graft S may then be introduced over the guidewire 150B and position the stent graft S through the first and second fistula to bypass the CTO, as shown in FIG. 3L. Alternatively, instead of a stent graft, two embolisation devices may be placed in the vein V, distal and proximal of the two fistulas to direct the flow of blood around the CTO.

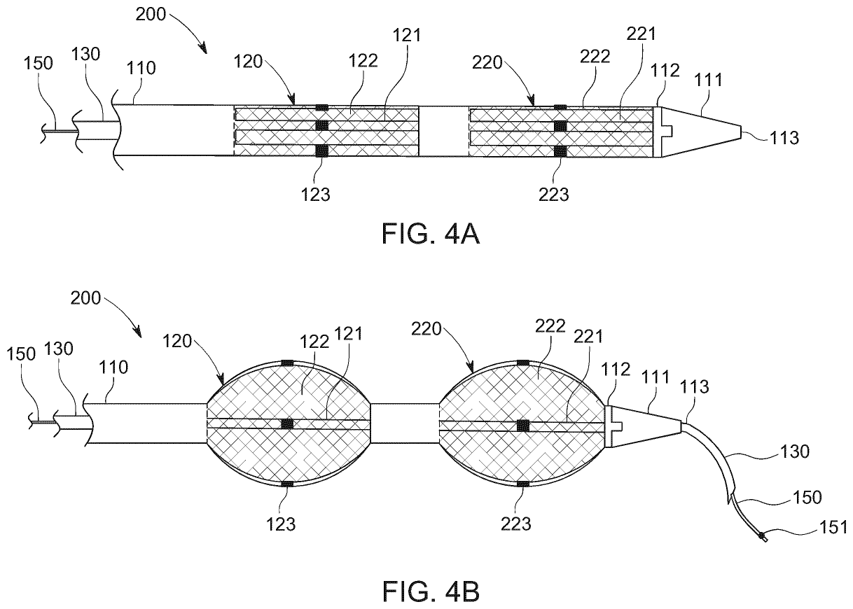

FIG. 4A shows an alternative embodiment of a crossing catheter 200. The crossing catheter 200 is similar to the crossing catheter 100 described in relation to FIGS. 1 and 2, above, and the same reference numerals are used to denote corresponding features.

The crossing catheter 200 differs from crossing catheter 100 in that it comprises a second stabilizing element 220 in addition to the stabilizing element 120. The second stabilizing element 220 may be disposed distally of the stabilizing element 120 with a small gap between the two. The second stabilizing element 220 may be identical to stabilizing element 120 and may comprises multiple flexible spines 221 covered by a mesh or screen 122. The second stabilizing element may comprise 3 to 10 flexible spines 221, or for example 3 to 5 flexible spines 221. Both stabilizing elements 120, 220 are in the contracted configuration in FIG. 4A.

FIG. 4B shows the crossing catheter 200 with the stabilizing element 120 and the second stabilizing element 220 in a laterally expanded configuration. The second stabilizing element 220 may be similar to stabilizing element 120 in that the flexible spines 221 bend to form a laterally expanded convex shape and allow the stabilizing element 220 to contact the inside of a vessel wall.

The addition of a second stabilizing element 220 to the crossing catheter 200, provides better stabilization to the crossing catheter 200 during the fistula formation process.

Furthermore, the second stabilizing element allows a guidewire 150 to be more easily captured or snared, as it allows for a greater margin of error when positioning the crossing catheter 200 to snare a guidewire 150.

The crossing catheter 200 may be used in the same way as crossing catheters 100A and 100B shown in FIGS. 3A to 3L.

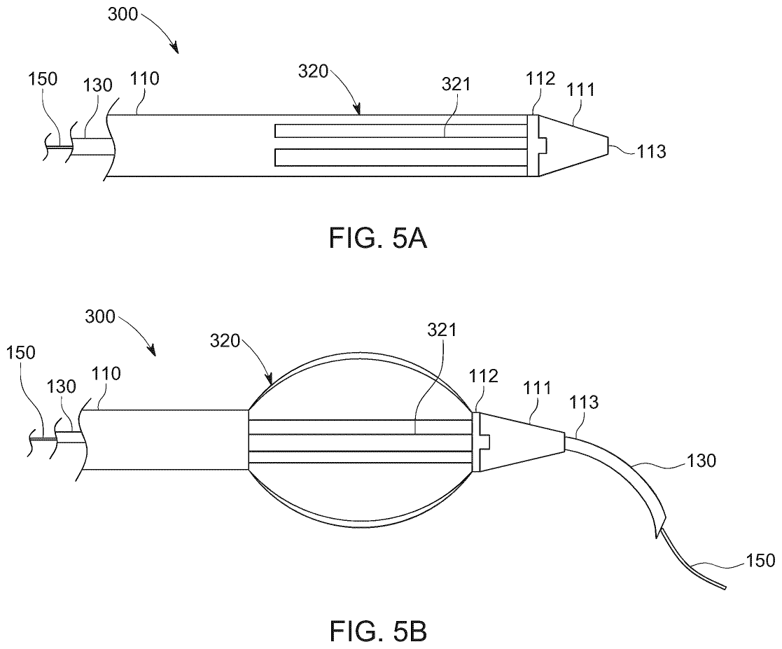

FIG. 5A shows another alternative embodiment of a crossing catheter 300. The crossing catheter 300 is similar to the crossing catheter 100 described in relation to FIGS. 1 and 2, above, and the same reference numerals are used to denote corresponding features.

The crossing catheter 300 differs from crossing catheter 100 in that the stabilizing element 320 does not comprise a mesh or screen and consists only of the flexible spines 321.

FIG. 5B shows the crossing catheter 300 with the stabilizing element 320 in a laterally expanded configuration. Similar to crossing catheter 100, in the laterally expanded configuration, the flexible spines 321 of the stabilizing element 320 may be bent into a convex shape such that they have a greater lateral or radial extent than in the contracted configuration. This allows the stabilizing element 320 to contact the inside of a vessel wall and thereby stabilize the crossing catheter 100. The stabilizing element 320 can also be used to dilate the diameter of a fistula as well as snare a guidewire.

The method of performing the endovascular bypass procedure of FIG. 3A-L can therefore also be performed with two of the crossing catheters 300 of FIG. 5.

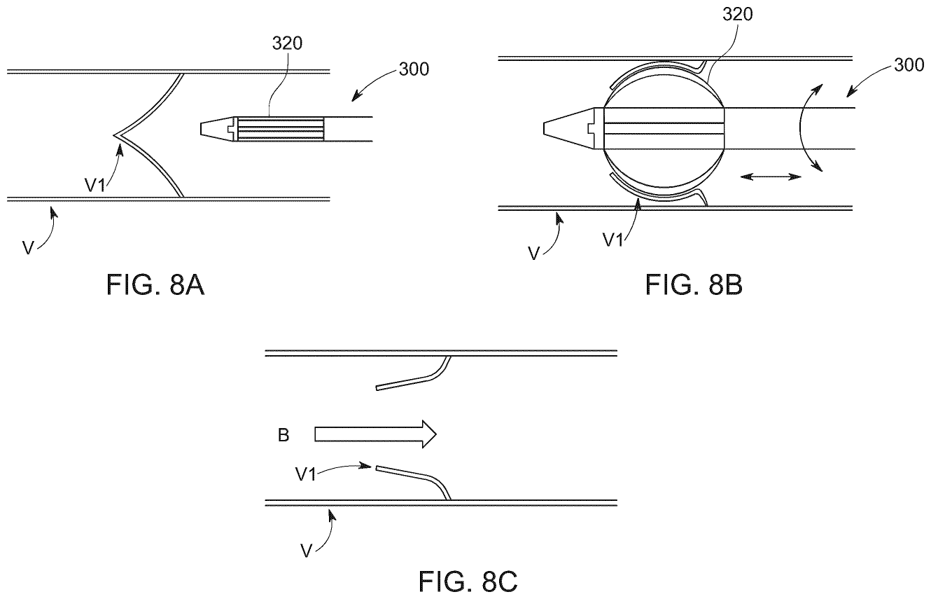

The advantage of having a crossing catheter 300 with no mesh or screen is that the stabilizing element can be used to better destroy or incapacitate a venous valve, as is described with respect to FIG. 8 below. Whilst this is also possible with a stabilizing element having a screen or mesh, removing the screen or mesh makes it easier to incapacitate the valves as the flexible spines 321 can come into direct contact with the valve.

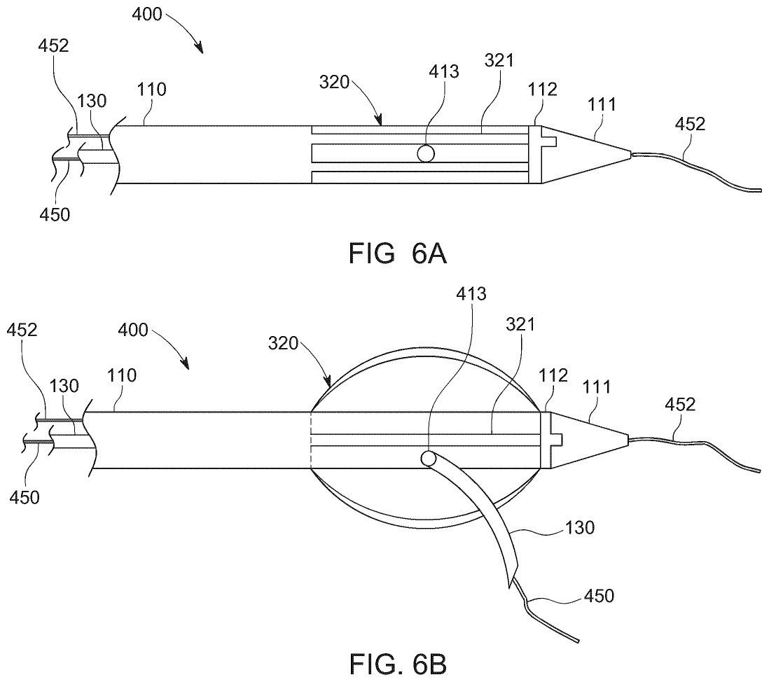

FIG. 6A shows another alternative embodiment of a crossing catheter 400. Again, the same reference numerals are used to denote corresponding features. Like the crossing catheter 300, this crossing catheter 400 also does not have a mesh or screen.

Furthermore, the crossing catheter 400 differs from that of FIG. 1 in that the penetration port 413 is not disposed at the distal end of the tapered distal tip 111. Rather, the penetration port 413 comprises a lateral opening which is disposed within the stabilizing element 320.

The crossing catheter 400 may be equipped with a mesh or screen, if the apertures of such a mesh or screen are made big enough to allow the penetration tool 130 to pass through.

FIG. 6B shows the stabilizing element 320 in a laterally expanded configuration with the penetration tool 130 in the advanced position where it extends out of the penetration port 413 and past the flexible spines 321. By having the penetration port 413 within the stabilizing element 320, this means that the tapered distal tip 111 can be advanced all the way to the CTO before forming a fistula.

The crossing catheter 400 comprises two guidewires 450 and 452. A first guidewire 450 is disposed within the lumen of the penetration tool 130 and can be advanced directly through the fistula formed by the penetration tool 130. The second guidewire 452 is disposed within the lumen of the shaft 110 and can extend out of the distal tip 111. This guidewire 452 can be used as a placement guidewire for conventional over-the-wire placement to guide the crossing catheter 400 to the appropriate treatment site.

Two of the crossing catheters 400 can also be used to perform the endovascular bypass procedure of FIGS. 3A-L. The method is essentially the same, except that the crossing catheter 400 cannot be immediately translated through the fistula after the fistula has been formed, as shown in FIG. 3D, for example. Rather the crossing catheter 400 must be removed from the artery A while the guidewire 450 remains in place and then placed over the guidewire 450 such that the distal tip 111 can follow the guidewire 450 and the crossing catheter 400 can be translated through the fistula.

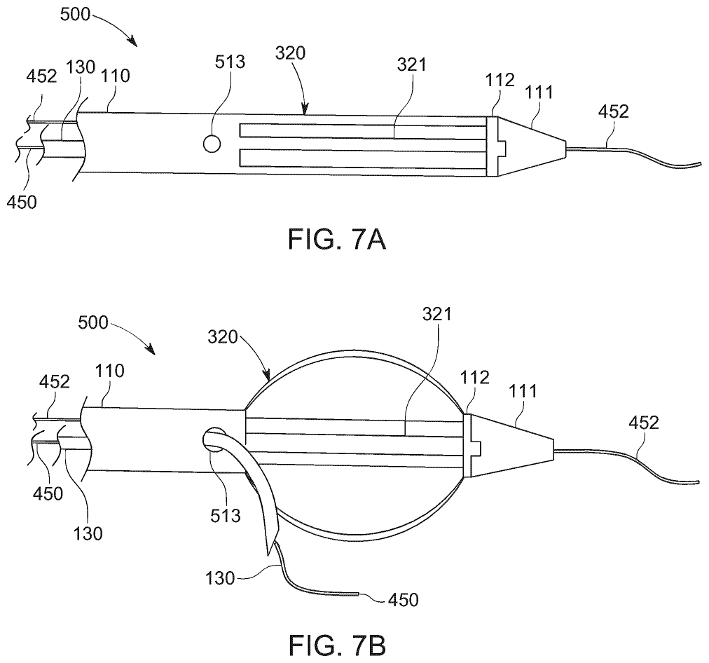

FIG. 7A shows another alternative embodiment of a crossing catheter 500. The same reference numerals are again used to denote corresponding features.

The crossing catheter 500 also does not have a mesh or screen and furthermore the penetration port 513 is placed proximally adjacent the stabilizing element 320. The advantage of this is that it allows the distal tip 111 to be advanced all the way to the CTO and the penetration tool 130 does not interfere with the stabilizing element 320. For example, if the stabilizing element comprises a larger number of flexible spines 321 or a mesh with smaller apertures, it may be difficult to place the penetration port within the stabilizing element 320.

The crossing catheter 500 may equally be equipped with a mesh or screen over the stabilizing element 320.

FIG. 7B shows the crossing catheter 500 with the stabilizing element 320 in the laterally expanded configuration with the penetration tool 130 in the advanced position where it extends out of the penetration port 513. The two guidewires 450 and 452 are also shown.

The radiopaque markers 123 and 152 are not shown in the crossing catheters of FIGS. 5 to 7, however, these embodiments may equally be equipped with such radiopaque markers.

FIGS. 8A to C illustrate a method of destroying a venous valve using the crossing catheter 300 of FIG. 5. However, the crossing catheters 400 and 500 of FIGS. 6 and 7 may equally be used to perform this method. The crossing catheters 100 and 200 can also be used, although, having no mesh or screen may allow the flexible spines 321 to better engage with the valve and therefore make destroying or incapacitating the valve more efficient.

As shown in FIG. 8A, the crossing catheter 300 is introduced into the vein V through a venous access site and advanced in a proximal direction. Once the crossing catheter 300 reaches a first valve v1, the distal tip 111 of the crossing catheter 300 is advanced through the valve v1 such that the stabilizing element 320 is in contact with the valve v1.

The stabilizing element 320 is then expanded into the laterally expanded configuration, as shown in FIG. 8B. The crossing catheter is then moved longitudinally back and forth while being rotated at the same time to incapacitate the valve v1.

Once the valve is incapacitated or destroyed, the stabilizing element 320 is collapsed into the contracted configuration and either moved to the next valve to be incapacitated or removed from the vein V.

The incapacitated valve v1 will allow blood B to flow in a distal direction, as shown in FIG. 8C.

FIGS. 9A to 9F illustrate a method of performing a deep vein arterialization procedure using two crossing catheters of FIG. 5, 300A and 300B.

In a deep vein arterialization procedure, a fistula is created between the artery and vein with the intent of “arterializing” the veins such that the blood can bypass a chronic total occlusion by flowing through the veins.

Firstly, a suitable access site is formed in a vein V, and a first guidewire 150A is introduced into the vein V through the venous access site. For a deep vein arterialization procedure, the venous access site may be distal of the CTO and the first direction D1 is therefore a proximal direction and the second direction D2 is a distal direction.

As shown in FIG. 9A, the first crossing catheter 300A is advanced to a first venous valve v1 which is incapacitated by rotating and simultaneously moving the expanded stabilizing element 320A back and forth. All of the other venous valves between the CTO and the venous access site are incapacitated in the same manner.

A second access site is then formed in the artery A proximal of the CTO and a second crossing catheter 300B is introduced into the artery and advanced in a second direction D2 to the CTO. The first crossing catheter 300A is advanced in a first direction D1 to the venous side of the predetermined crossing point. As shown in FIG. 9B, the stabilizing element 320A of the first crossing catheter 300A is then expanded into the laterally expanded configuration to stabilize the first crossing catheter 300A in the vein V, opposite the predetermined crossing point.

The stabilizing element 320B of the second crossing catheter 120B is then expanded to stabilize the crossing catheter 300B for the fistula formation process. The guidewire 150B is retracted and the penetration tool 130B is advanced such that it extends out of the penetration port 113B. The sharp distal needle end of the penetration tool 130B deflects laterally and penetrates the vessel wall of the artery A and vein V to form a fistula between the artery A and vein V, proximal of the CTO.

The guidewire 150B is advanced through the guidewire lumen in the penetration tool 130B and through the fistula directly into the vein V. The expanded stabilizing element 320A of the first crossing catheter 300A is positioned in the vein V next to the second fistula and the guidewire 150B is advanced into and past the stabilizing element 320A.

The stabilizing element 320A is then collapsed to snare the guidewire 150B, as shown in FIG. 9C. The first crossing catheter 300A together with guidewire 150B is then pulled back in a second direction D2 and out of the venous access site.

The penetration tool 130B of the second crossing catheter 300B is then retracted, the stabilizing element 320B is collapsed and the second crossing catheter 300B is translated through the fistula.

As shown in FIG. 9D, when the stabilizing element 320B is positioned within the fistula, the stabilizing element 320B is expanded to dilate the size of the fistula. The stabilizing element 320B is collapsed again and the second crossing catheter 300B is then pulled back in a first direction D1 such that only the guidewire 150B is left, as illustrated in FIG. 9E. The guidewire 150B extends from the arterial access site through the fistula into the vein V, past the CTO and out of the venous access site.

A standard balloon catheter with a stent graft S may then be introduced over the guidewire 150B to position the stent graft S through the fistula to bypass the CTO, as shown in FIG. 9F. Alternatively, instead of a stent graft, an embolisation devices may be placed in the vein V, proximal to the fistula to direct the flow of blood past the CTO.

The crossing catheters 100, 200, 400 or 500 may also be used to perform this deep vein arterialization procedure.

The method would be the same for crossing catheters 100 and 200, although a separate valve destroying catheter could be used if a more efficient destruction of the valves is required.

For the crossing catheters 400 and 500, the second crossing catheter 400B, 500B would need to be removed from the artery between steps of FIGS. 9C and 9D, and the second crossing catheter 400B, 500B would be placed over the guidewire 150B before it is advanced through the fistula.

The present disclosure therefore provides a new type of catheter system, which can perform a deep vein arterialization or endovascular bypass procedure in a simpler and more efficient manner, requiring fewer steps and fewer catheters. The new type of catheter system can also be flexibly utilised to cross between arteries and veins at different points to bypass a CTO.

Various modifications will be apparent to those skilled in the art.

The stabilizing element 120 may not comprise any radiopaque markers 123.

The stabilizing element 120 may not comprise a mesh or screen 122. The mesh or screen 122 may not have an antithrombic coating.

The stabilizing element 120 may not comprise any flexible spines 122, but rather may comprise a different type of expandable cage such as an expandable braided structure, for example.

The crossing catheter 100 may not comprise the radiopaque marker 112.

The crossing catheter 100 may not comprise a handle 140.

The crossing catheter 100 may not comprise a guidewire lumen or guidewire 150.

The guidewire 150 may not comprise a radiopaque marker 151. The distal tip 111 may not have a tapered shape but may be cylindrical, for example. The crossing catheter 100 may not comprise a distal tip 11.

The penetration port 113 may be a longitudinal opening, a lateral opening or disposed at an angle to the longitudinal axis.

The crossing catheter may comprise more than two stabilizing elements. The two or more stabilizing elements of the crossing catheter may not be identical.

The two crossing catheters 100A, 100B may not be identical.

For example, one of the two crossing catheters may have two or more stabilizing elements.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter set for forming a fistula between two adjacent blood vessels, the catheter set comprising:
two crossing catheters, each crossing catheter comprising:
a catheter shaft defining a longitudinal axis and having a distal end section;
a stabilizing element disposed in the distal end section of the catheter shaft; and
a penetration tool for penetrating vessel walls of two adjacent blood vessels to form the fistula, wherein the penetration tool is disposed within the catheter shaft and is movable between a retracted position and an advanced position;
wherein the stabilizing element comprises an expandable cage having a contracted configuration and a laterally expanded configuration, and
wherein the expandable cage is adapted to snare a guidewire when moving from the laterally expanded configuration to the contracted configuration.

2. The catheter set of claim 1, wherein at least one of the stabilizing elements further comprises a mesh or a screen which covers the expandable cage.

3. (canceled)

4. The catheter set of claim 1, wherein at least one of the expandable cages comprises a plurality of flexible spines.

5. The catheter set of claim 1, wherein at least one of the expandable cages comprises a radiopaque marker.

6. (canceled)

7. The catheter set of claim 1, wherein at least one of the two crossing catheters comprises a guidewire lumen for accommodating a guidewire.

8. The catheter set of claim 7, wherein the guidewire lumen is disposed within the penetration tool, such that the guidewire lumen allows placement of the guidewire through the fistula formed by the penetration tool.

9.-11. (canceled)

12. The catheter set of claim 1, wherein each catheter shaft comprises a penetration lumen for accommodating the penetration tool.

13. The catheter set of claim 12, wherein the distal end of each penetration lumen ends in a penetration port through which the penetration tool can exit the crossing catheter.

14.-15. (canceled)

16. The catheter set of claim 13, wherein at least one of the penetration ports is disposed adjacent the stabilizing element.

17. The catheter set of claim 13, wherein at least one of the penetration ports is disposed within the stabilizing element.

18. The catheter set of claim 13, wherein the stabilizing element is a first stabilizing element and at least one of the crossing catheters further comprises a second stabilizing element.

19. The catheter set of claim 18, wherein the penetration port is disposed between the first stabilizing element and the second stabilizing element.

20. The catheter set of claim 18, wherein the second stabilizing element comprises an expandable cage having a contracted configuration and a laterally expanded configuration.

21.-22. (canceled)

23. The catheter set of claim 1, wherein a distal end of at least one of the penetration tools deflects laterally as the penetration tool is moved distally from the retracted position to the advanced position.

24. (canceled)

25. A crossing catheter for forming a fistula between two adjacent blood vessels, comprising:
a catheter shaft defining a longitudinal axis and having a distal end section;
a stabilizing element disposed in the distal end section of the catheter shaft; and
a penetration tool for penetrating vessel walls of the two adjacent blood vessels to form the fistula, wherein the penetration tool is disposed within the catheter shaft and is movable between a retracted position and an advanced position;
wherein the stabilizing element comprises an expandable cage having a contracted configuration and a laterally expanded configuration, and
wherein the expandable cage is adapted to snare a guidewire when moving from the laterally expanded configuration to the contracted configuration.

26. The crossing catheter of claim 25, wherein the stabilizing element further comprises a mesh or a screen which covers the expandable cage, and wherein the expandable cage comprises a plurality of flexible spines.

27.-49. (canceled)

50. A method for bypassing a total chronic occlusion in a blood vessel using a first and second crossing catheter, each crossing catheter comprising a catheter shaft defining a longitudinal axis and having a distal end section, a stabilizing element comprising an expandable cage disposed in the distal end section of the catheter shaft, and a penetration tool disposed within the catheter shaft, the method comprising:
introducing the first crossing catheter into the blood vessel comprising the total chronic occlusion from a first side;
advancing the first crossing catheter to a first position adjacent the chronic total occlusion;
stabilizing the first crossing catheter by moving the stabilizing element of the first crossing catheter from a contracted configuration to a laterally expanded configuration;
forming a first fistula between the blood vessel and an adjacent blood vessel by moving the penetration tool of the first crossing catheter from a retracted position to an advanced position and penetrating vessel walls between the two adjacent blood vessels;
moving the stabilizing element of the first crossing catheter from the laterally expanded configuration to the contracted configuration;
translating the first crossing catheter through the first fistula into the adjacent blood vessel;
introducing the second crossing catheter into the blood vessel comprising the total chronic occlusion from a second side;
advancing the second crossing catheter to a second position adjacent the chronic total occlusion on the opposite side to the first position;
moving the stabilizing element of the first crossing catheter from a contracted configuration to a laterally expanded configuration;
stabilizing the second crossing catheter by moving the stabilizing element of the second crossing catheter from a contracted configuration to a laterally expanded configuration;
forming a second fistula between the blood vessel and the adjacent blood vessel by moving the penetration tool of the second crossing catheter from a retracted position to an advanced position and penetrating vessel walls between the two adjacent blood vessels;
passing a guidewire through the second fistula from the first blood vessel to the second blood vessel;
snaring the guidewire with the stabilizing element of the first crossing catheter by moving from the laterally expanded configuration to the contracted configuration;
pulling the first crossing catheter and the guidewire through the first fistula into the blood vessel.

51. The method of claim 50, wherein translating the first crossing catheter through the first fistula further comprises:
expanding the size of the first fistula by moving the stabilizing element of the first crossing catheter from a contracted configuration to a laterally expanded configuration.

52. The method of claim 50, wherein translating the second crossing catheter through the second fistula further comprises:
expanding the size of the second fistula by moving the stabilizing element of the second crossing catheter from a contracted configuration to a laterally expanded configuration.
